(19) United States
(12) Patent Application Publication
(10) Pub. No.: US 2026/0091012 A1
(43) Pub. Date: Apr. 2, 2026

(54) Methods Of Treating Meduloblastoma With Thyroid Hormone

(71) Applicant: Institute For Cancer Research d/b/a The Research Institute of Fox Chase Cancer Center, Philadelphia, PA (US)

(72) Inventors: Zengjie Yang, Philadelphia, PA (US); Yijun Yang, Philadelphia, PA (US)

(73) Assignee: Institute For Cancer Research d/b/a The Research Institute of Fox Chase Cancer Center, Philadelphia, PA (US)

(21) Appl. No.: 19/114,982

(22) PCT Filed: Sep. 21, 2023
(86) PCT No.: PCT/US2023/074763

§ 371 Date: Mar. 25, 2025

Related U.S. Application Data
(60) Provisional application No. 63/409,802, filed on Sep. 25, 2022.

(51) Int. Cl. A61K 31/198; A61K 45/06; A61P 35/00

(52) U.S. Cl. CPC A61K 31/198; A61K 45/06; A61P 35/00

(57) ABSTRACT

The present disclosure provides methods of treating a medulloblastoma in a subject in need comprising administering T3 to the subject. The present disclosure also provides methods of inhibiting proliferation of medulloblastoma cells in a subject in need by administering T3 to the subject. Moreover, the present disclosure provides uses of T3 for treating medulloblastoma and the manufacture of a medicament for treating medulloblastoma.

### FIELD

The present disclosure is directed, in part, to methods of treating medulloblastoma with thyroid hormone.

### BACKGROUND

A medulloblastoma is a malignant pediatric brain tumor that arises in the cerebellum, a part of the brain located at the base of the skull. This tumor is the most common malignant brain tumor in children; it commonly strikes children between 5 and 9 years of age and is rare in people over 30. Treatment includes surgery, radiation, and chemotherapy. Treatment for medulloblastoma focuses on removing as much of the tumor as safely possible and relieving pressure in the child's skull (e.g., intracranial pressure) due to swelling or hydrocephalus. In addition to surgical removal of the tumor, a physician may sometimes recommend a shunt to help drain cerebrospinal fluid buildup and steroid treatments to reduce tumor swelling. Surgery is often followed by radiation and chemotherapy. These therapies address cancer cells that might have been unreachable by surgery and those that have spread from the tumor to other parts of the brain or spinal cord. Medulloblastoma spread and recurrence is common; radiation and chemotherapy can reduce the risks.

Despite the aggressive tumor treatment including surgical resection, chemotherapy and radiation, a significant proportion of patients with medulloblastoma still succumbs to this disease. Moreover, patients who survive medulloblastoma, often suffer severe side effects of aggressive treatment, such as endocrine disorder and cognitive deficit. More effective and less toxic approaches to treat medulloblastoma are urgently needed.

Thyroid hormones triiodothyronine (T3) and thyroxine (T4) are two hormones produced and released by follicular cells of the thyroid gland. Both T3 and T4 are tyrosine-based hormones largely responsible for the regulation of metabolism. Thyroid hormones are essential for proper development and differentiation of all cells influencing a variety of physiological and pathological processes such as increasing basal metabolic rate, affecting protein synthesis, regulating lone bone growth, increasing catecholamine sensitivity, regulating protein, fat, and carbohydrate metabolism, stimulating vitamin metabolism, and inhibiting neuronal activity. Several disorders may arise from both an excess and a deficiency of thyroid hormone including hyperthyroidism (Graves' disease), hypothyroidism (Hashimoto's thyroiditis), clinical depression, hair loss, and cardiovascular disorders.

### SUMMARY

The present disclosure provides methods of treating a medulloblastoma in a subject in need thereof, the method comprising administering T3 to the subject.

The present also disclosure provides methods of inhibiting proliferation of medulloblastoma cells in a subject in need thereof, the methods comprising administering T3 to the subject.

The present disclosure also provides methods of inhibiting differentiation of medulloblastoma cells in a subject in need thereof, the methods comprising administering T3 to the subject.

The present disclosure also provides uses of T3 for treating medulloblastoma.

The present disclosure also provides use of T3 in the manufacture of a medicament for treating medulloblastoma.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows T3 inhibition of MB cell proliferation; MB cells were treated with different concentration of T3 or T3 antagonist 1-850 for 48 hours (Panel A); the percentage of EdU+ cells in the culture were quantified; DAPI was used to counterstain cell nuclei (Panel B).

FIG. 2 shows T3 induced MAP2 expression in MB cells.

FIG. 3 shows survival curves of MB ptch^{-/-} tumor mice with or without T3 treatment (Panel A); NeuN and pHH3 expression in paraffin embed sections of mouse MB by immunohistochemistry (Panel B).

FIG. 4 (Panel A, Panel B, and Panel C) shows T3 inhibition of human shh type MB growth.

FIG. 5 shows survival curves of SmoM2 tumor mice with or without T3 treatment, vismodegib (GDC) as a control (Panel A); NeuN and pHH3 expression in paraffin embed sections of mouse MB by immunohistochemistry (Panel B).

FIG. 6 shows T3 inhibition of MB group3(Ms) and Human MB group3 cell lines proliferation; Group3 (Ms)MB cells were treated with T3 or T3 antagonist 1-850 for 72 hours; the percentage of EdU+ cells in the culture were quantified (Panel A); D283 were treated with T3 or T3 antagonist 1-850 for 72 hours; the percentage of EdU+ cells in the culture were quantified (Panel B); D341 were treated with T3 or T3 antagonist 1-850 for 72 hours; the percentage of EdU+ cells in the culture were quantified (Panel C).

FIG. 7 shows that cisplatin inhibited cell proliferation significantly (except 31.23 nM and 62.5 nM) and MB cell proliferation was decreased by T3 (Panel A); and MB cells treated with T3 combined with cisplatin at two different concentrations showed a synergistic effect (Panel B).

FIG. 8 shows that T3 treatment significantly repressed tumor progression in the recipient mice (Panel A), and reduced luciferase levels in mouse brains compared with PBS treatment (Panel B); the survival of recipient mice was significantly prolonged after T3 treatment (Panel C), as compared with PBS treatment (median survival: T3 treatment, undefined vs PBS treatment, 15 days; p<0.01).

### DESCRIPTION OF EMBODIMENTS

Unless defined otherwise, all technical and scientific terms have the same meaning as is commonly understood by one of ordinary skill in the art to which the embodiments disclosed belongs.

As used herein, the terms “a” or “an” means that “at least one” of “one or more” unless the context clearly indicates otherwise.

As used herein, the term “about” means that the numerical value is approximate and small variations would not significantly affect the practice of the disclosed embodiments. Where a numerical value can vary ±10% and remain with the scope of the disclosed embodiments.

As used herein, the terms “comprising” (and any form of comprising, such as “comprise”, “comprises” and “comprised”), “having” (and any form of having, such as “have” and “has”), “including” (and any form of including, such as “contains” and “contain”), are inclusive or open-ended and do not exclude additional, unrecited elements of method steps.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. For example, a subject who receives treatment with T3, as described herein, in order to treat medulloblastoma is “in need thereof” (i.e., as opposed to receiving T3 to treat hypothyroidism).

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal of a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophosphate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. Salts also includes quaternary ammonium salts of the compounds described herein, where the compounds have one or more tertiary amine moiety.

As used herein, the terms “treat,” “treated,” or “treating” mean therapeutic treatment wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

Recently, it has been discovered (as described herein) that many medulloblastoma patients have hypothyroidism, a condition in which the thyroid gland does not produce enough thyroid hormone. Moreover, patients with medulloblastoma also frequently develop hypothyroidism, as a side effect of tumor treatment, particularly after radiotherapy. These observations led to the examination, as described herein, of the possible functions of thyroid hormone in medulloblastoma growth. In the studies described herein, the proliferation of medulloblastoma cells was significantly inhibited by treatment with thyroid hormone, suggesting that thyroid hormone could be used to treat medulloblastoma.

The present disclosure provides methods of treating a medulloblastoma in a subject in need thereof, the method comprising administering T3 to the subject.

The present disclosure also provides methods of inhibiting proliferation of medulloblastoma cells in a subject in need thereof, the method comprising administering T3 to the subject.

The present disclosure also provides methods of inhibiting differentiation of medulloblastoma cells in a subject in need thereof, the methods comprising administering T3 to the subject.

The present disclosure also provides uses of T3 for treating medulloblastoma.

The present disclosure also provides uses of T3 in the manufacture of a medicament for treating medulloblastoma.

In any of the methods described herein the T3 can be commercially obtained. In some embodiments, the T3 is liothyronine, triostat, or LEVO-T®, LEVOXYL®, NOVOTHYROX®, SYNTHROID®, and UNITHROID® (levothyroxine sodium). In some embodiments, the T3 is levothyroxine sodium.

In any of the embodiments described herein, the subject may not have hypothyroidism. In any of the embodiments described herein, the subject may have hypothyroidism, but is receiving treatment with T3 to treat medulloblastoma.

In some embodiments, the T3 is present in amount from about 0.1 mg to about 250 mg, from about 1 mg to about 100 mg, from about 5 mg to about 50 mg, from about 7.5 mg to about 40 mg, or from about 10 mg to about 30 mg. In some embodiments, the T3 is present in amount from about 5 mg to about 100 mg, from about 5 mg to about 90 mg, from about 5 mg to about 80 mg, from about 5 mg to about 70 mg, from about 5 mg to about 60 mg, from about 5 mg to about 50 mg, from about 5 mg to about 40 mg, from about 5 mg to about 30 mg, from about 5 mg to about 20 mg, or from about 5 mg to about 10 mg. In some embodiments, the T3 is present in amount from about 5 mg to about 50 mg, from about 1 mg to about 50 mg, from about 5 mg to about 50 mg, from about 10 mg to about 50 mg, from about 15 mg to about 50 mg, from about 20 mg to about 50 mg, from about 25 mg to about 50 mg, from about 30 mg to about 50 mg, from about 35 mg to about 50 mg, from about 40 mg to about 50 mg, or from about 45 mg to about 50 mg.

In some embodiments, the subject is a child up to 17 years of age. In some embodiments, the subject is an adult at least 18 years of age.

In some embodiments, the medulloblastoma is Wnt subtype, Hh subtype, group 3 subtype, or group 4 subtype. In some embodiments, the medulloblastoma is Wnt subtype. In some embodiments, the medulloblastoma is Hh subtype. In some embodiments, the medulloblastoma is group 3 subtype. In some embodiments, the medulloblastoma is group 4 subtype.

In any of the embodiments described herein, the subject can also be administered a chemotherapeutic agent. Examples of chemotherapeutic agents include, but are not limited to, cisplatin, carboplatin, oxaliplatin, nedaplatin, triplatin tetranitrate, phenanthriplatin, picoplatin, satraplatin, methotrexate, vincristine, doxorubicin, tunicamycin, oligomycin, bortezomib, MG132, 5-flurouracil, sorafenib, flavopiridol, gemcitabine, taxol, mercaptopurine, thioguanine, hydroxyurea, cytarabine, mitomycin, cyclophosphamide, ifosfamide, nitrosourea, dacarbazine, procarbazine, an etoposide, a campothecin, bleomycin, idarubicin, daunorubicin, dactinomycin, distamycin A, ethidium, netropsin, auristatin, amsacrine, prodigiosin, bortezomib, pibenzimol, tomaymycin, duocarmycin SA, plicamycin, mitoxantrone, asparaginase, vinblastine, vinorelbine, paclitaxel, docetaxel, CPT-11, gleevec, erlotinib, gefitinib, ibrutinib, crizotinib, ceritinib, lapatinib, navitoclax, vismodegib, temozolomide, and regorafenib, or any combination thereof. In some embodiments, the chemotherapeutic agent is a combination of agents, such as, for example, methotrexate/vincristine/doxorubicin/cisplatin (MVAC) or gemcitabine/cisplatin. In any of the embodiments described herein, the subject can be administered a combination of T3 and cisplatin.

In any of the embodiments described herein, the subject can also be administered an immunotherapeutic agent. Examples of immunotherapeutic agents include, but are not limited to, OPDIVO® (nivolumab), KEYTRUDA® (pembrolizumab), TECENTRIQ® (atezolizumab), IMFINZI® (durvalumab), YERVOY® (ipilumumab), ERBITUX® (cetuximab), AVASTIN®, (bevacizumab), HERCEPTIN® (trastuzumab), PERJETA® (pertuzumab), VECTIBIX® (panitumumab), PORTRAZZA™ (necitumumab), UNITUXIN™ (dinutuximab), CIRAMZA® (ramucirumab), LARTRUVO® (olaratumab), KADCYLA® (ado-trastuzumab emtansineb), XGEVA® (denosumab), and BAVENCIO® (avelumab), or any combination thereof. In some embodiments, the immunotherapeutic agent is nivolumab, pembrolizumab, atezolizumab, durvalab, ipilumumab, cetuximab, bevacizumab, trastuzumab, pertuzumab, panitumumab, necitumumab, dinutuximab, ramucirumab, olaratumab, ado-trastuzumab emtansineb, denosumab, or avelumab, or any combination thereof. In some embodiments, the immunotherapeutic agent is nivolumab, pembrolizumab, atezolizumab, durvalab, ipilumumab, cetuximab, bevacizumab, or trastuzumab, or any combination thereof. In some embodiments, the immunotherapeutic agents include immune checkpoint inhibitors. Examples of immune checkpoint inhibitors include, but are not limited to, YERVOY® (Ipilimumab), tremelimumab, MGA271, MGA271, indoximod, INCB024360, BMS-986016, or any combination thereof. In some embodiments, the immunotherapeutic agents include PD-1 and/or PD-L1 inhibitors. Examples of PD-1 and PD-L1 inhibitors include but are not limited to OPDIVO® (nivolumab), KEYTRUDA® (pembrolizumab), TECENTRIQ® (atezolizumab), BAVENCIO® (avelumab). IMFINZI® (durvalumab), LIBTAYO® (cemiplimab) JTX-4014, spartalizumab, camrelizumab, sintilimab, tislelizumab, toripalimab, dostarlimab, pidilizumab, INCMGA00012, AMP-224, AMP-514, KN035, CK-301, AUNP12, CA-170, and BMS986189, or any combination thereof.

In any of the embodiments described herein, the subject can also be administered radiation therapy.

In some embodiments, the pharmaceutical composition is an oral dosage formulation, an intravenous dosage formulation, a topical dosage formulation, an intraperitoneal dosage formulation, or an intrathecal dosage form.

In some embodiments, the pharmaceutical composition is an oral dosage formulation in the form of a pill, tablet, capsule, cachet, gel-cap, pellet, powder, granule, or liquid.

In some embodiments, the pharmaceutical composition the oral dosage formulation is protected from light and present within a blister pack, bottle, or intravenous bag.

The compounds and compositions described herein can be formulated for parenteral administration by injection, such as by bolus injection or continuous infusion. The compounds and compositions can be administered by continuous infusion subcutaneously over a period of about 15 minutes to about 24 hours. The compositions can take such forms as suspensions, solutions or emulsions in oily or aqueous vehicles, and can contain formulary agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds and compositions described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds and compositions described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

In transdermal administration, the compounds and compositions can be applied to a plaster, or can be applied by transdermal, therapeutic systems that are consequently supplied to the organism. In some embodiments, the compounds and compositions are present in creams, solutions, powders, fluid emulsions, fluid suspensions, semi-solids, ointments, pastes, gels, jellies, and foams, or in patches containing any of the same.

The compounds and compositions described herein can also be formulated as a depot preparation. Such long-acting formulations can be administered by implantation (for example subcutaneously or intramuscularly) or by intramuscular injection. Depot injections can be administered at about 1 to about 6 months or longer intervals. Thus, for example, the compounds and compositions can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds and compositions can be delivered in a controlled release system. In some embodiments, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In some embodiments, a controlled-release system can be placed in proximity of the target of the compounds and compositions described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

The compounds and compositions described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

In some embodiments, the compounds and compositions described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The amount of compound to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

The compounds and compositions described herein can be administered by any route of administration including, but not limited to, oral, sublingual, buccal, rectal, intranasal, inhalation, eye drops, ear drops, epidural, intracerebral, intracerebroventricular, intrathecal, epicutaneous or transdermal, subcutaneous, intradermal, intravenous, intraarterial, intraosseous infusion, intramuscular, intracardiac, intraperitoneal, intravesical infusion, and intravitreal. In some embodiments, the administration is oral, sublingual, buccal, rectal, intranasal, inhalation, eye drops, or ear drops. In some embodiments, the administration is oral, sublingual, buccal, rectal, intranasal, or inhalation. In some embodiments, the administration is epidural, intracerebral, intracerebroventricular, or intrathecal. In some embodiments, the administration is epicutaneous or transdermal, subcutaneous, or intradermal. In some embodiments, the administration is intravenous, intraarterial, intraosseous infusion, intramuscular, intracardiac, intraperitoneal, intravesical infusion, or intravitreal. In some embodiments, the administration is intravenous, intramuscular, or intraperitoneal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, it may be desirable to administer one or more compounds, or a pharmaceutically acceptable salt thereof, or compositions comprising the same, to a particular area in need of treatment. This may be achieved, for example, by local infusion (for example, during surgery), topical application (for example, with a wound dressing after surgery), by injection (for example, by depot injection), catheterization, by suppository, or by an implant (for example, where the implant is of a porous, non-porous, or gelatinous material, including membranes, such as silastic membranes, or fibers). Formulations for injection can be presented in unit dosage form, such as in ampoules or in multi-dose containers, with an added preservative.

The present disclosure also provides pharmaceutical compositions comprising T3 for use in the manufacture of a medicament for treating medulloblastoma.

The present disclosure also provides uses of pharmaceutical compositions comprising T3 for reducing treating medulloblastoma.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: General Methodology

### Mice:

Math1-Cre mice, Ptch1^{fl/fl} mice, R26-SmoM2 mice, Math1-CreER mice, CAG-Cas9 mice and were purchased from the Jackson Laboratory. All mice were bred and genotyped as recommended by the Jackson Laboratory. CB17/SCID mice were bred in the Fox Chase Cancer Center Laboratory Animal Facility (LAF).

### Cell Culture:

MB cells were isolated from Math1-Cre/Ptch1^{fl/fl}, Math1-Cre/Ptch1^{fl/fl}/Cas9 and SmoM2 tumor-bearing mice at 6 to 8 weeks of age, respectively. Tumor tissue was dissociated in a papain solution to obtain a single-cell suspension and then centrifuged through a 35% and 65% Percoll gradient. Cells from the 35% to 65% interface were suspended in Dulbecco's PBS (DPBS) plus 0.5% BSA. Cells were then suspended in NB-B22 (B22 supplement followed protocol in Jacob Hanna's lab) and plated on poly-D-lysine (PDL)-coated coverslips (BD Biosciences). D283, D341 and MB-shh cell lines were cultured in DMEM with 10% fetal bovine serum, 1% Pen/Strep and 2 mM L-glutamine (Invitrogen).

### Drug Treatment:

For examining in vitro efficacies, the cells were cultured with or without T3 for 48 hours in NB-B22 culture medium. For examining in vivo efficacies, T3 (200 ng/g) in DPBS and administrated to mice by i.p. injection twice a day. GDC0449 (50 mg/kg) diluted using MCT and administrated to mice by oral gavage once a day. For survival analyses, mice after treatment with T3 or GDC0449 were monitored daily. Log-rank survival analyses were performed in GraphPad Prism 9.

EdU(5-ethynyl-2′-deoxyuridine) Incorporation and Cell Immunostaining:

The EdU incubated cells were cultured at 1 μM in culture medium for 1 hour. 4% PFA was used to fix the cells for 10 minutes. The cells were incubated with the staining solution for 20 minutes in the dark. The cells were washed with PBST for 5 minutes/time for 3 times.

Immunofluorescent staining of cells was carried out according to standard methods. Briefly, cells were blocked and permeabilized for 1 hour with PBS containing 0.1% Triton X-100 and 10% normal goat serum, stained with primary antibodies (1:400) overnight at 4° C. and incubated with secondary antibodies (1:500) for 1 hour. Cells were counterstained with DAPI and mounted with Fluoromount-G (Southern Biotech) before being visualized using a Nikon Eclipse Ti microscope.

### Tumor Transplantation:

MB cells were stereotaxically injected into the cerebellum of 6- to 8-week-old SCID mice. Before transplantation, MB cells were infected with lentivirus carrying firefly luciferase for 24 hours. For transplantation with MB cells after the virus infection, the cell suspension was centrifuged at 1000 rpm for 5 minutes to remove the supernatant. 1×10⁶ cells (based on Trypan blue staining) were injected into the cerebellum of each mouse. Animals were monitored weekly using in vivo bioluminescence imaging.

### Example 2: T3 Inhibits MB Cell Proliferation

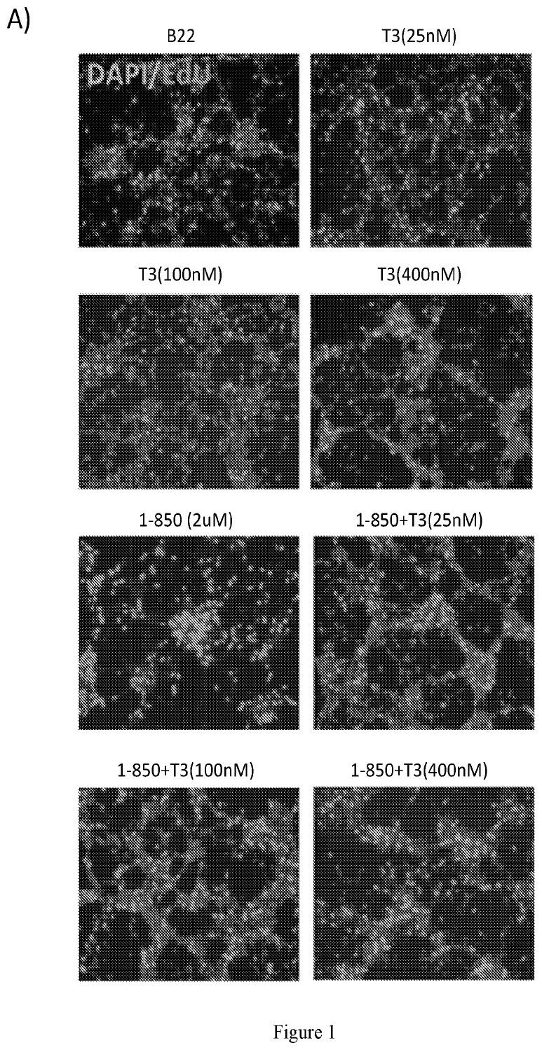

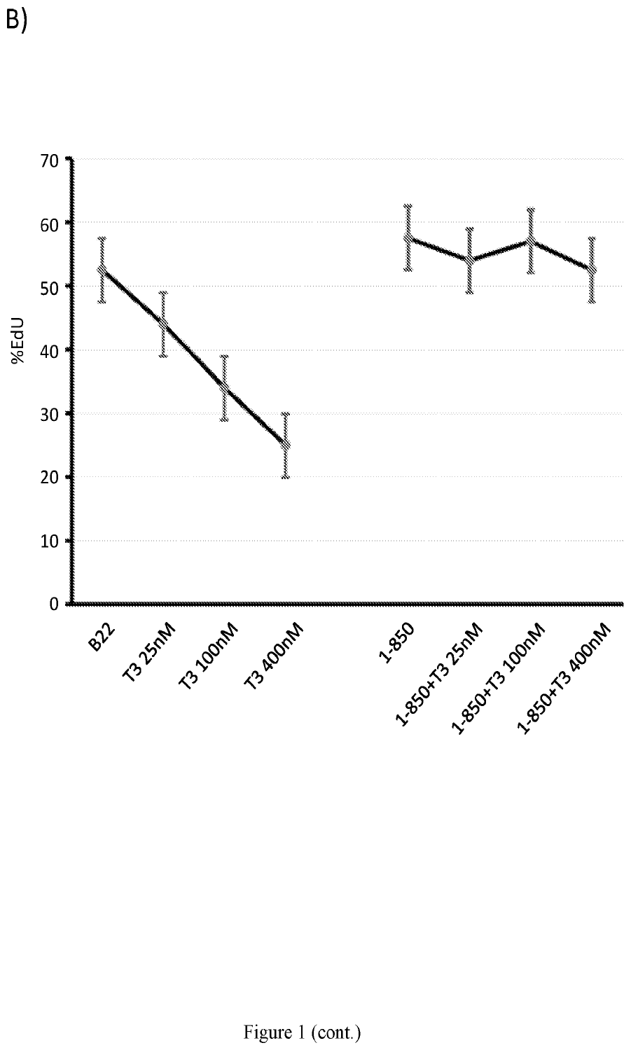

To examine the function of T3 in MB cells, supplement B22 that is T3 free was prepared. Recent MB culture medium supplement B27 include T3. NB-B22 cultured MB ptch^{-/-} cells were treated with different concentrations of T3 for 48 hours (see, FIG. 1, Panel A). Also, when T3 antagonist 1-850 was added in to the medium, the results showed that the proliferation of medulloblastoma cells was significantly inhibited by treatment with T3 and rescued by 1-850. The inhibition was concentration dependent. The percentage of EdU+ cells in the culture were quantified (see, FIG. 1, Panel B). DAPI was used to counterstain cell nuclei. Thus, T3 repressed tumor cells proliferation through the thyroid hormone receptor (TR) pathway.

### Example 3: T3 Induced MB Cell Differentiation

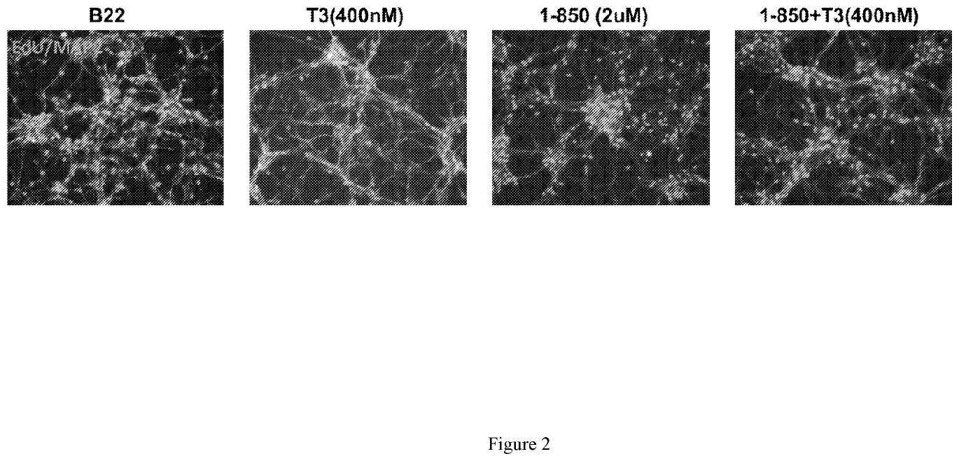

To determine whether T3 was able to induce MB cell differentiation, immunostaining was used to detect differentiation marker MAP2 expression in MB that were treated by T3 for 48 hours. The results shown that T3 increased MAP2 expression (see, FIG. 2). Thus, T3 induced MB cell differentiation and repressed tumor proliferation. Therefore, blocking the TR pathway can abolish T3 inhibited function on tumor cells.

### Example 4: T3 Prolonged the Math1-Cre/Ptch1^{fl/fl} Mice Survival and Induced Tumor Cell Differentiation

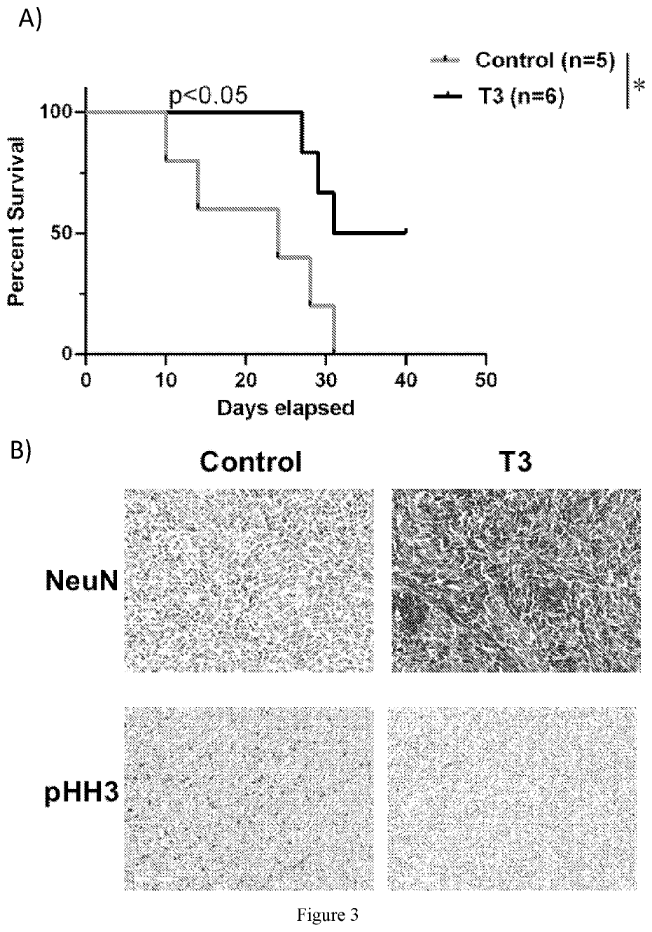

Math1-Cre/Ptch1^{fl/fl} tumor mice were treated by T3, and without T3 as a control. T3 prolonged the Math1-Cre/Ptch1^{fl/fl} mice survival significantly (see, FIG. 3, Panel A). NeuN, a differentiation marker, showed enhanced expression in T3-treated mice tumor tissues. Conversely, pHH3, a cell proliferation maker, was decreased in T3-treated mice tumor tissues. These results suggest that T3 inhibits cell proliferation through inducing cell differentiation. In addition, T3 repressed tumor progression in vivo.

### Example 5: T3 Inhibited Human shh Type MB Growth

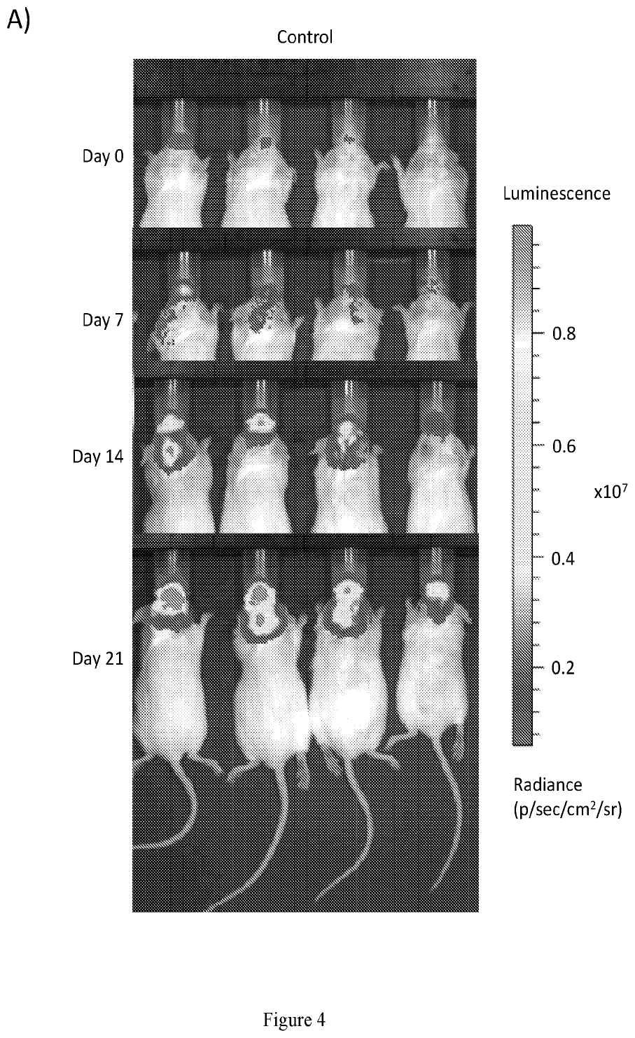

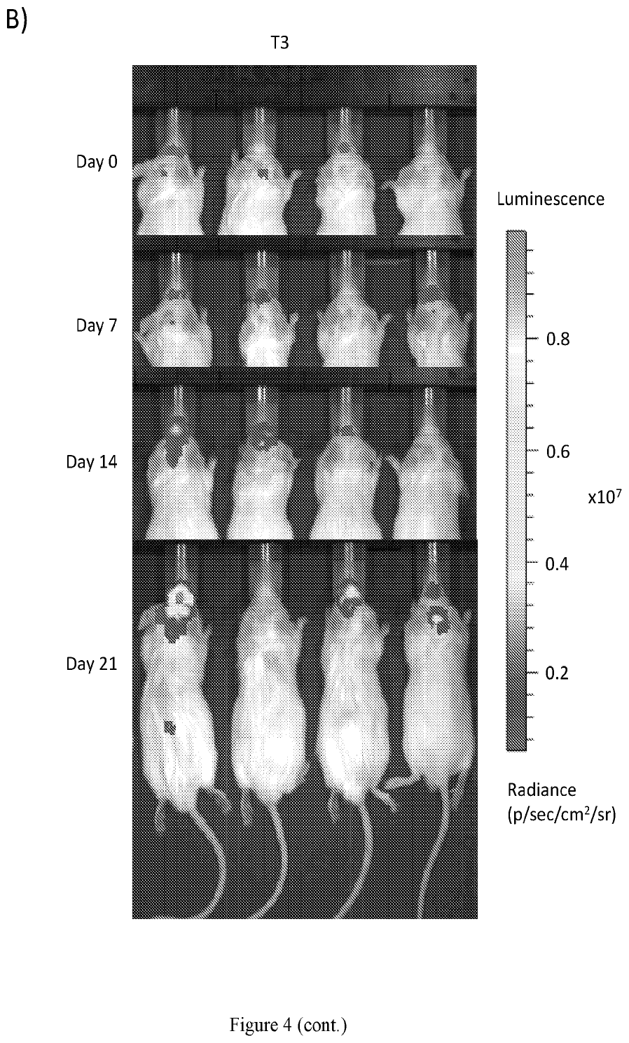

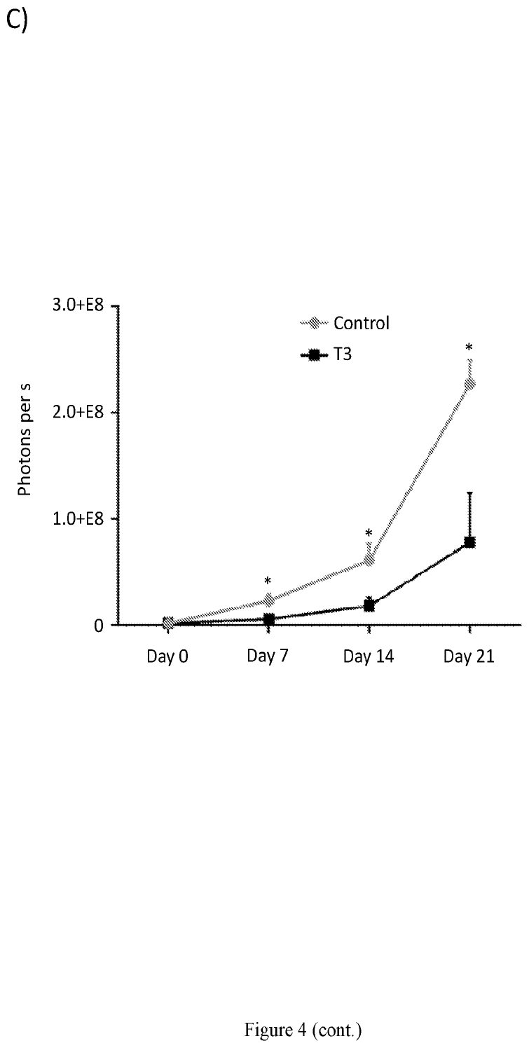

Shh type PDX-luciferase cells were generated. Those cells were stereotaxically injected into the cerebellum of 6- to 8-week-old SCID mice. Animals were monitored weekly using in vivo bioluminescence imaging and then bioluminescence were detected as Day 0. The mice were untreated controls (see, FIG. 4, Panel A) or treated by T3 for 3 weeks (see, FIG. 4, Panel B). T3 significantly inhibited MB growth (see, FIG. 4, Panel C).

### Example 6: T3 Inhibited SmoM2 MB Tumor Growth and Prolonged SmoM2 Mice Survival

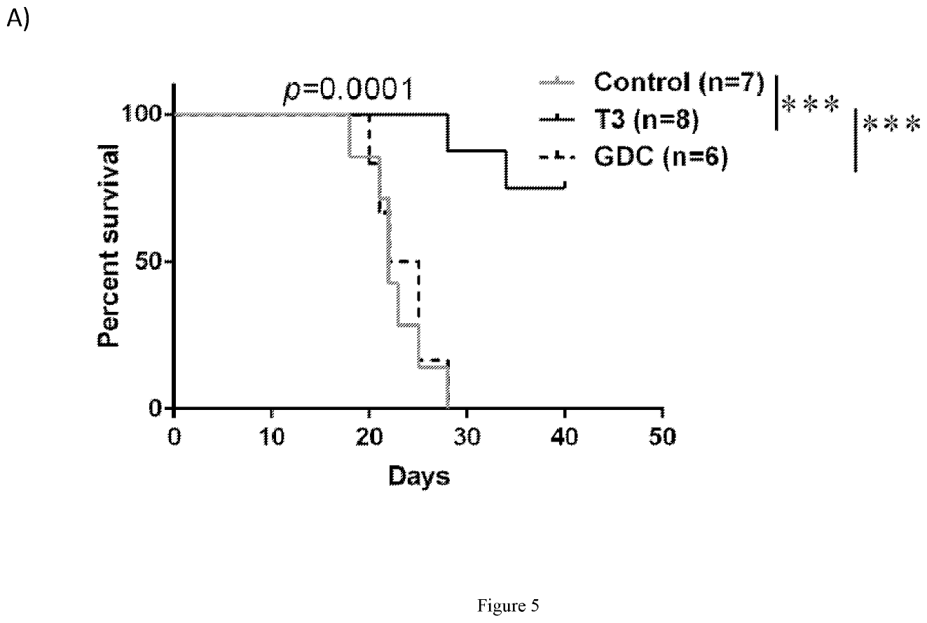

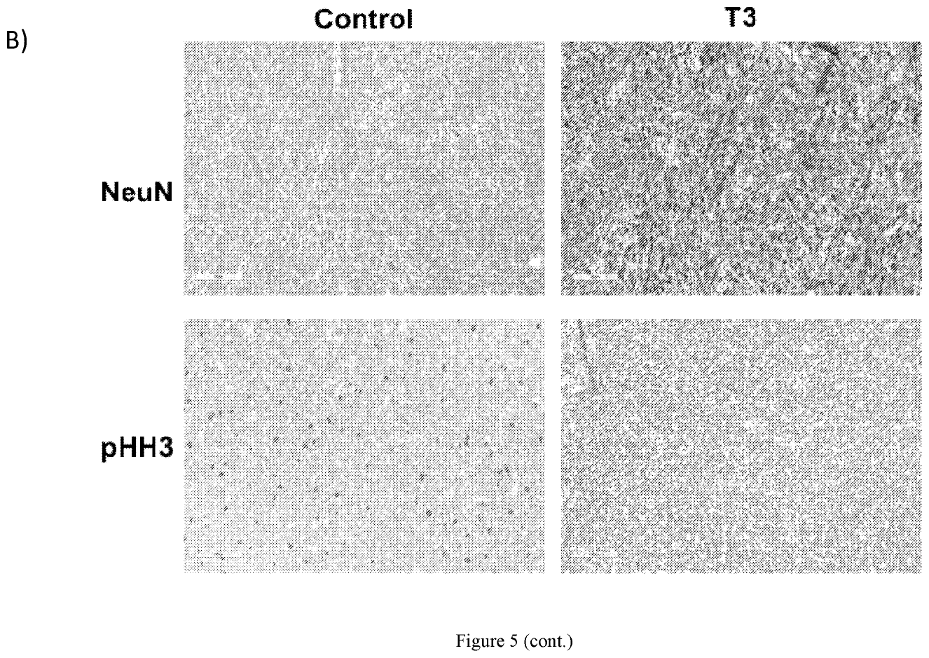

SmoM2 tumor mice were treated by T3, without T3 and vismodegib (GDC) as control. T3 prolonged the SmoM2 mice survival significantly (see, FIG. 5, Panel A). NeuN, a differentiation marker, showed enhanced expression in T3-treated mice tumor tissues. Conversely, pHH3, a cell proliferation maker, was decreased in T3-treated mice tumor tissues (see, FIG. 5, Panel B). Overall, the results indicate that T3 induced differentiation overrides oncogenic mutation.

### Example 7: T3 Inhibited MB Group3 (Mouse) and Human MB Group3 Cell Line Proliferation

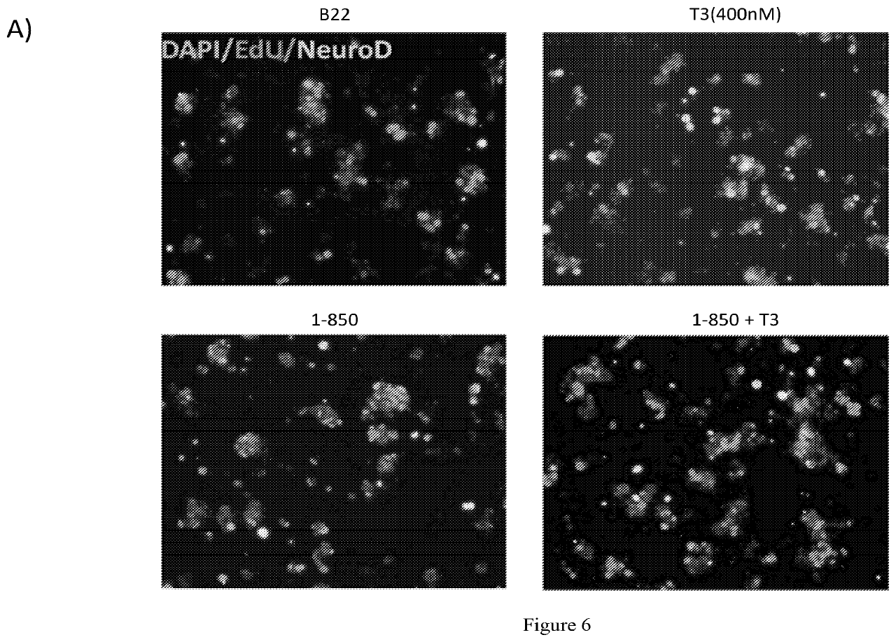

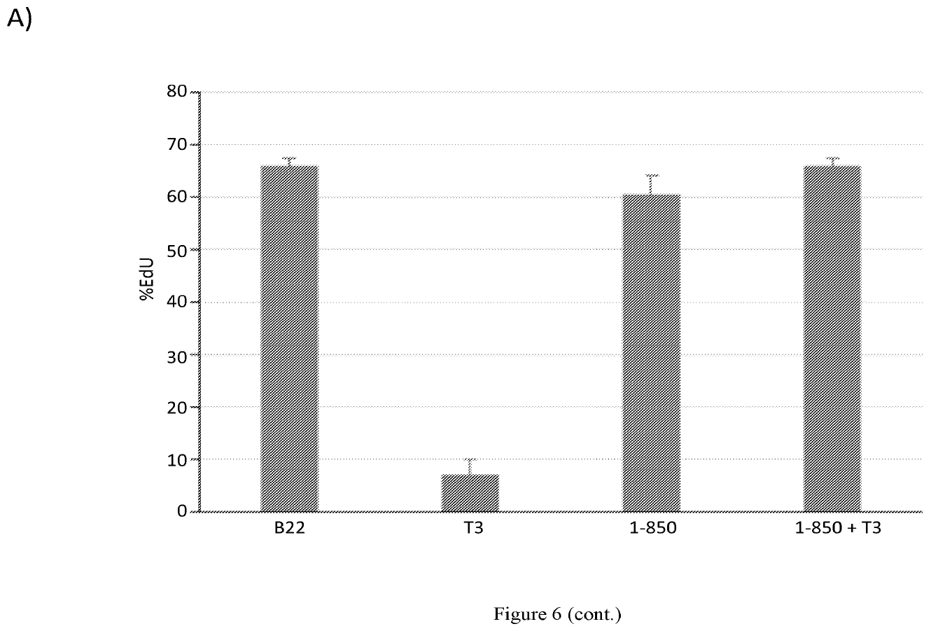

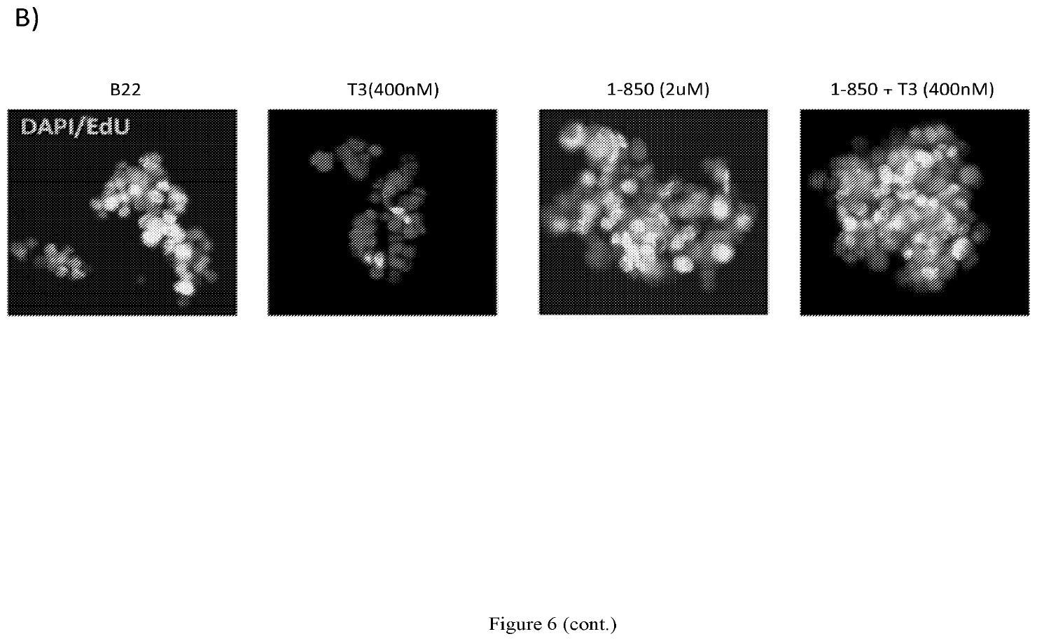

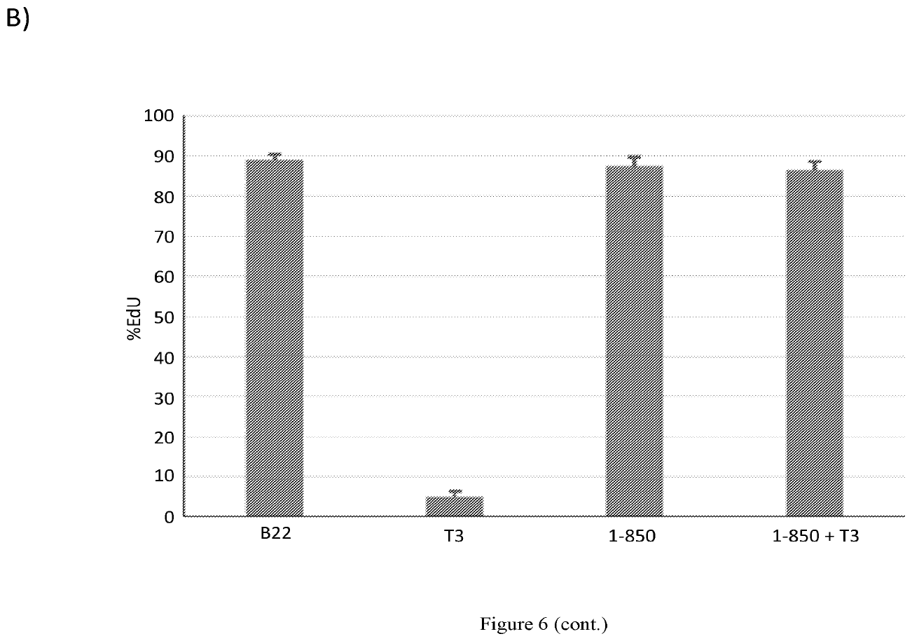

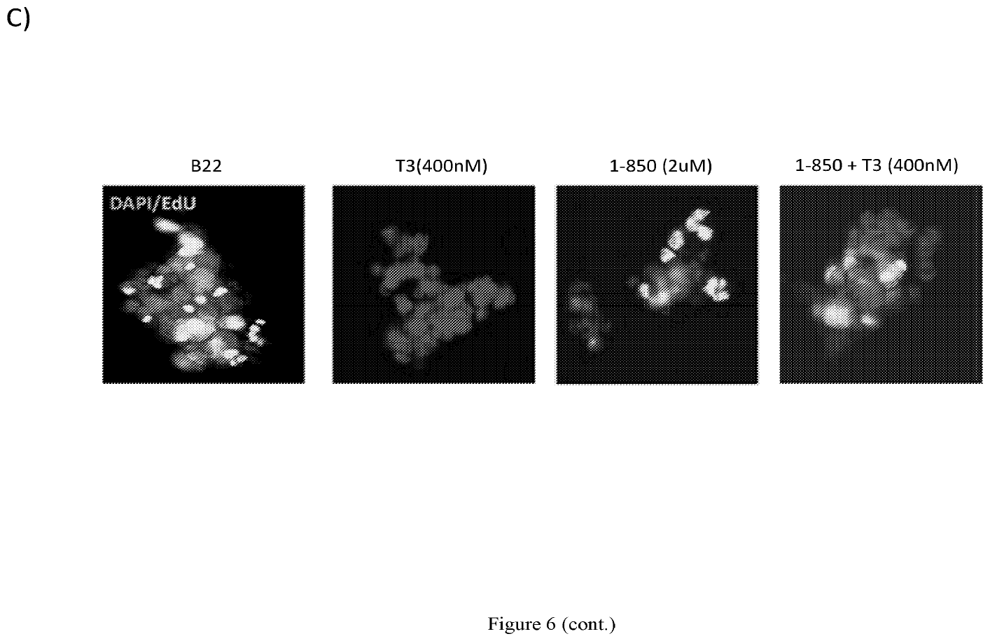

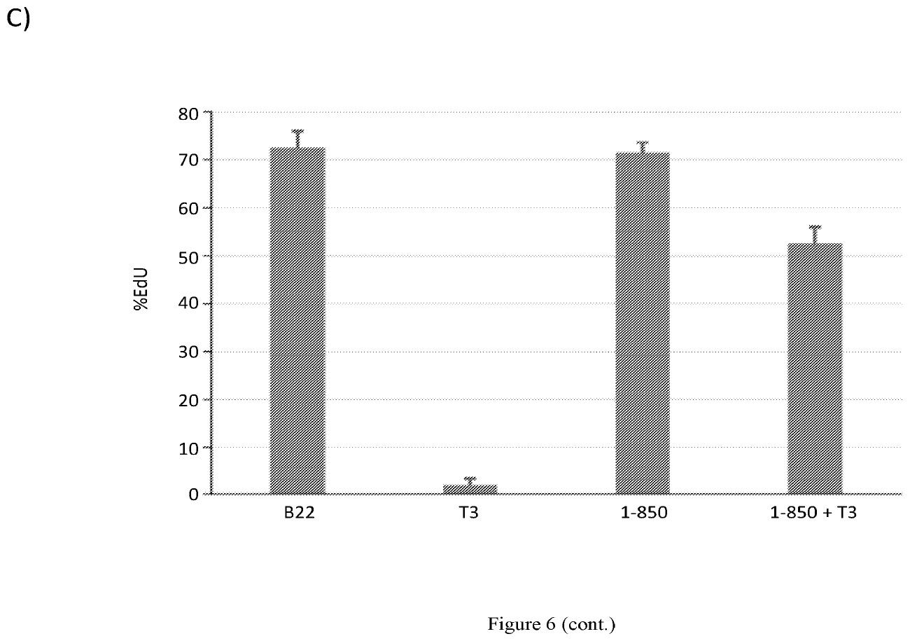

In order to examine the function of T3 in different types of MB, NB-B22 cultured MB group3 (Mouse) and Human MB group3 cell lines (D283 and D341) were treated with T3 for 72 hours. Group3 (Ms)MB cells were treated with T3 or T3 antagonist 1-850 for 72 hours. The percentage of EdU+ cells in the culture was quantified (see, FIG. 6, Panel A). D283 were treated with T3 or T3 antagonist 1-850 for 72 hours. The percentage of EdU+ cells in the culture was quantified (see, FIG. 6, Panel B). In addition, D341 cells were treated with T3 or T3 antagonist 1-850 for 72 hours. The percentage of EdU+ cells in the culture were quantified (see, FIG. 6, Panel C). Also, when T3 antagonist 1-850 was added to the medium, the results showed that the proliferation of group3 tumor cells were significantly inhibited by T3 and rescued by 1-850. These results suggest that T3 inhibits not only shh type MB growth, but also group3 MB. Thus, thyroid hormone may be used to treat different type of medulloblastoma.

### Example 8: T3 Synergizes with Cisplatin in Inhibiting MB Cell Proliferation

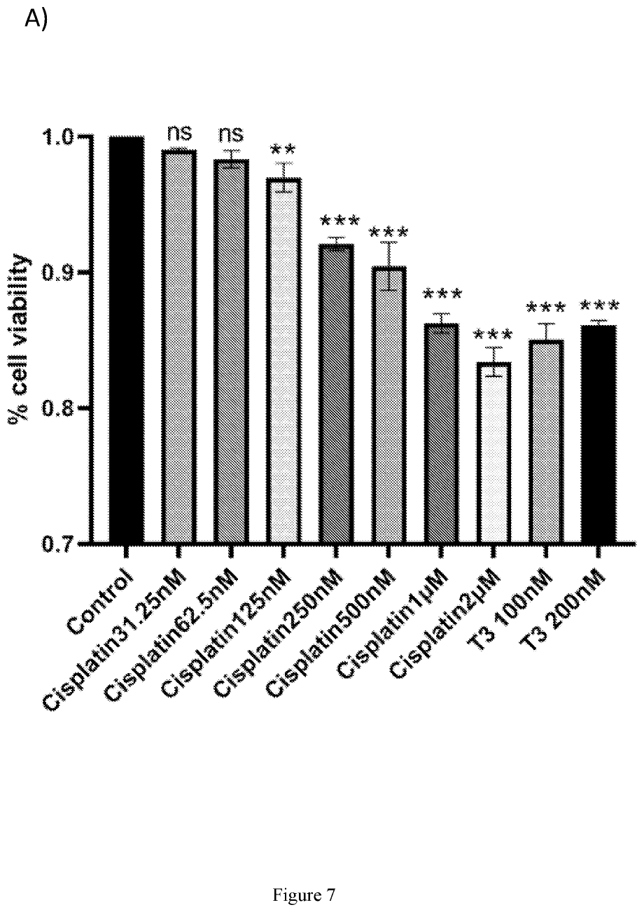

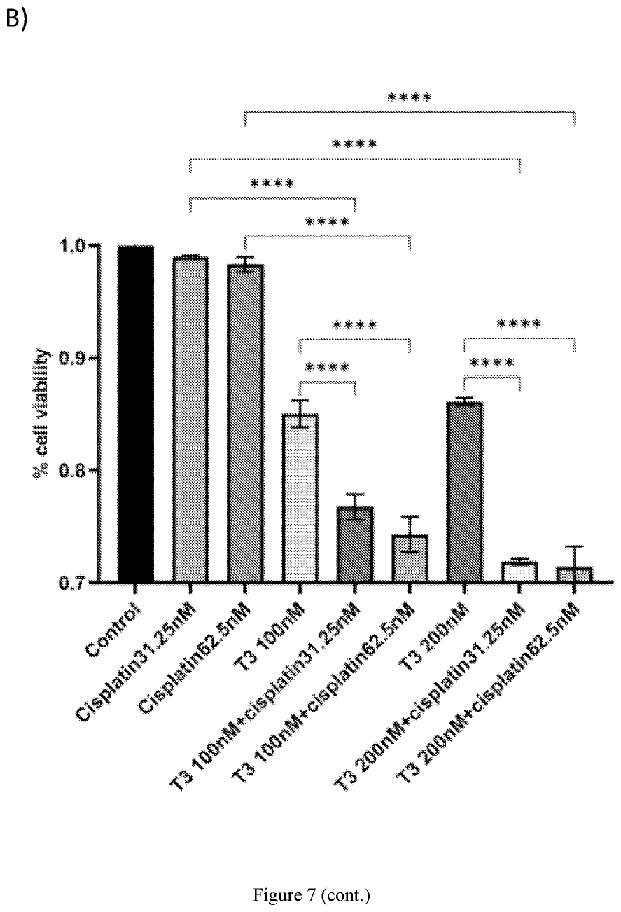

MB cells were isolated from Math1-Cre/Ptch1^{fl/fl} tumor mice and were treated in vitro with T3 at two concentrations. MB cells were treated with vehicle (DMSO) as a control. Cisplatin, a potent chemotherapeutic agent used in standard MB protocol, treated the cells at seven different concentrations. The survival of MB cells was measured by Cell Counting Kit-8 (CCK8) following the treatment. FIG. 7, Panel A shows cisplatin inhibited cell proliferation significantly except 31.23 nM and 62.5 nM of cisplatin. MB cell proliferation was decreased by T3 treatment as well. MB cells were also treated with T3 combined with cisplatin at two different concentration (FIG. 7, Panel B). The synergistic effects were found in the combination of T3 and cisplatin. Both 31.23 nM and 62.5 nM of cisplatin combined with T3 inhibited cell proliferation compared with cisplatin treatment alone and T3 treatment alone.

### Example 9: T3 Inhibits the Pathogenicity of Human G3-MB

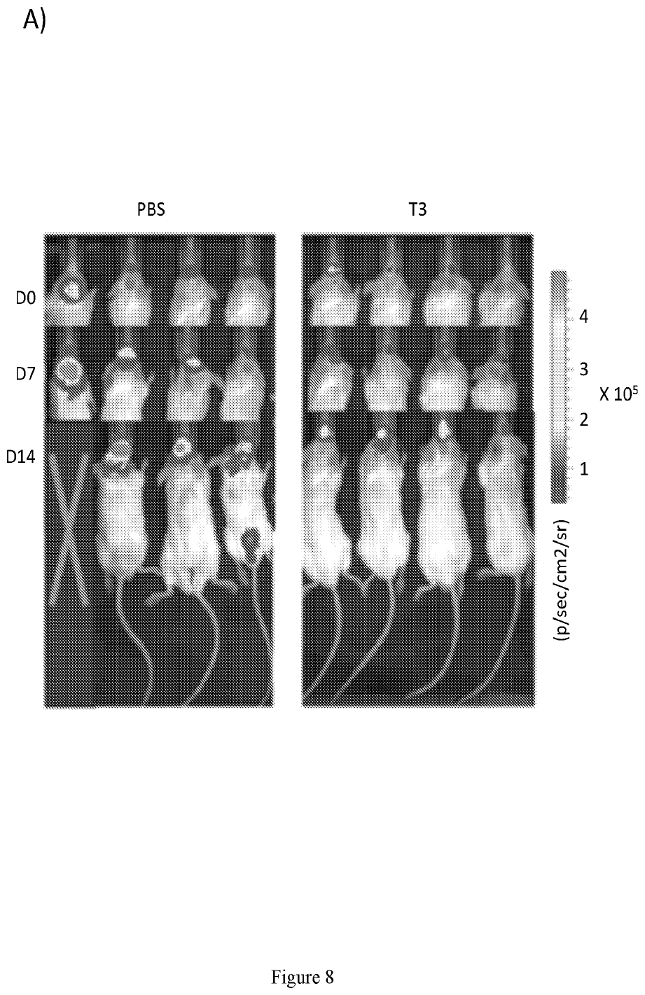

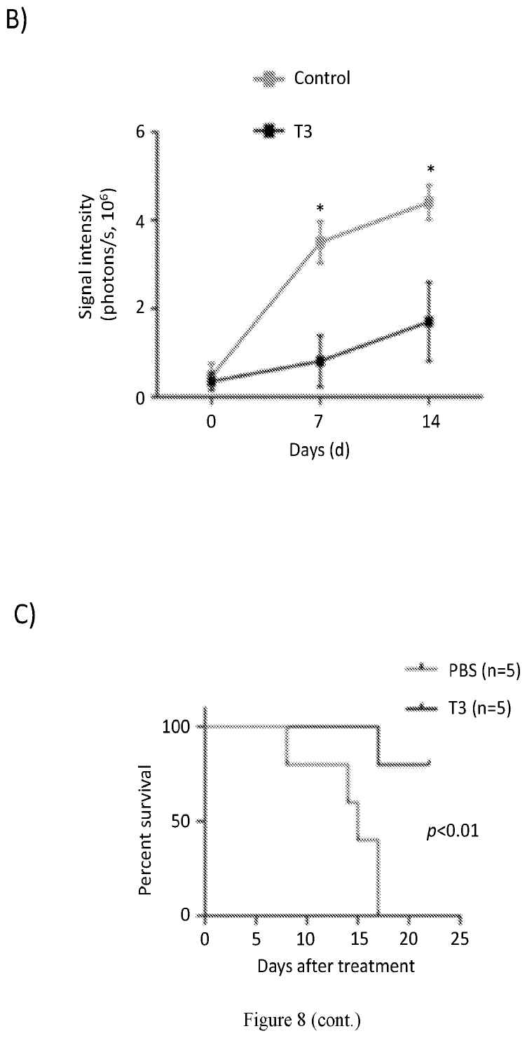

G3-MB PDOXs were generated by intracranial transplantation of RCMB28 cells transduced with a luciferase expressing construct. After tumors were established, mice were treated with T3 or PBS by i.p. injection. T3 treatment significantly repressed tumor progression in the recipient mice (FIG. 8, Panel A), and reduced luciferase levels in mouse brains compared with PBS treatment (FIG. 8. Panel B). The survival of recipient mice was significantly prolonged after T3 treatment (FIG. 8, Panel C), as compared with PBS treatment (median survival: T3 treatment, undefined vs PBS treatment, 15 days; p<0.01).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method of treating a medulloblastoma in a subject in need thereof, the method comprising administering thyroid hormone (T3) to the subject.

2. The method of claim 1, wherein the subject does not have hypothyroidism.

3. The method of claim 1, wherein the subject is also administered a chemotherapeutic agent.

4. The method of claim 1, wherein the subject is also administered an immunotherapeutic agent.

5. The method of claim 1, wherein the subject is also administered radiation therapy.

6. A method of inhibiting proliferation of medulloblastoma cells in a subject in need thereof, the method comprising administering thyroid hormone (T3) to the subject.

7. The method of claim 6, wherein the subject does not have hypothyroidism.

8. The method of claim 6, wherein the subject is also administered a chemotherapeutic agent.

9. The method of claim 6, wherein the subject is also administered an immunotherapeutic agent.

10. The method of claim 6, wherein the subject is also administered radiation therapy.

11. A method of inhibiting differentiation of medulloblastoma cells in a subject in need thereof, the method comprising administering thyroid hormone (T3) to the subject.

12. The method of claim 11, wherein the subject does not have hypothyroidism.

13. The method of claim 11, wherein the subject is also administered a chemotherapeutic agent.

14. The method of claim 11, wherein the subject is also administered an immunotherapeutic agent.

15. The method claim 11, wherein the subject is also administered radiation therapy.

16. Use of thyroid hormone (T3) for treating medulloblastoma.

17. Use of thyroid hormone (T3) in the manufacture of a medicament for treating medulloblastoma.
